(19) United States
(12) Patent Application Publication
(10) Pub. No.: US 2017/0209403 A1
(43) Pub. Date: Jul. 27, 2017

(54) METHOD FOR REGULATING ALDEHYDE DEHYDROGENASE 1

(71) Applicants: National Cheng Kung University, Tainan City (TW); National Cheng Kung University Hospital, Tainan (TW)

(72) Inventors: Cheng-Yang Chou, Tainan City (TW); Yi-Hui Wu, Tainan City (TW); Yu-Fang Huang, Tainan City (TW)

(21) Appl. No.: 15/008,053

(22) Filed: Jan. 27, 2016

(51) Int. Cl. A61K 31/203

(52) U.S. Cl. CPC A61K 31/203

(57) ABSTRACT

The present invention provides a method for regulating aldehyde dehydrogenase 1 (ALDH1) comprises administering all-trans retinoic acid to a subject. Further, the present invention also provides a method for treating solid malignancy comprises administering all-trans retinoic acid to a subject, providing a new choice in current cancer treatment.

### BACKGROUND OF THE INVENTION

1. Field of the Invention

The present invention relates to a method for regulating catalyze, especially relates to a method for regulating aldehyde dehydrogenase 1 (ALDH1).

2. The Prior Arts

Globally, as of 2010, about 160,000 people died from ovarian cancer, up from 113,000 in 1990. As of 2014, more than 220,000 diagnoses of epithelial ovarian cancer were made yearly. In 2010, in the United States, an estimated 21,880 new cases were diagnosed and 13,850 women died of ovarian cancer. Ovarian cancer is the second most common gynaecological malignancy and a major cause of death from cancer in women. Epithelial ovarian cancer (EOC) is usually diagnosed at an advanced stage, and despite cytoreductive surgery followed by combination chemotherapy, many EOC patients eventually experience recurrence with the development of chemoresistant tumours and subsequently die of their disease.

Fallopian tube cancer (often just tubal cancer) is thought to be a relatively rare primary cancer among women accounting for 1 to 2 percent of all gynecologic cancers. In the USA, tubal cancer had an incidence of 0.41 per 100,000 women from 1998 to 2003. Demographic distribution is similar to ovarian cancer, and the highest incidence was found in white, non-Hispanic women and women aged 60-79.

Primary peritoneal cancer is a cancer of the cells lining the peritoneum, or abdominal cavity. Some studies indicate that up to 15% of serous ovarian cancers are thought to be actually primary peritoneal carcinomas in origin.

Growing evidence implicates tubal, ovarian, and so-called primary peritoneal carcinomas as having a common origin, pathogenesis, and behavior. Ovarian cancer cell lines are usually used for in-vitro and animal studies for these three cancers. Regarding the treatment, the NCCN Guidelines discuss fallopian tube cancer and primary peritoneal cancer that are managed in a similar manner to epithelial ovarian cancer. In the clinic, these cancers are treated with the same chemotherapeutic agents even when they recur after primary therapy. Additionally, clinical trials for ovarian cancer are commonly designed to enroll patients with these three cancers. Despite the current clinical therapeutics for these three cancers are significantly extend the patient's lifetime, there is still room for improvement.

### SUMMARY OF THE INVENTION

To solve the problem described above, the present invention provides a method for regulating ALDH1 comprises administering an effective dose of all-trans retinoic acid to a subject.

In one embodiment of the present invention, the all-trans retinoic acid further regulates FoxM1 and Notch1 expression through regulating ALDH1.

In one embodiment of the present invention, the subject is a mammal. In a preferred embodiment of the present invention, the mammal is a human body and the effective dose is at least 0.00405 mg/kg.

The present invention also provides a method for treating solid malignancy comprises administering an effective dose of all-trans retinoic acid to a subject.

In one embodiment of the present invention, the subject is a mammal. In a preferred embodiment of the present invention, the mammal is a human body and the effective dose is at least 0.00405 mg/kg.

In one embodiment of the present invention, the solid malignancy is ovarian cancer, fallopian tube cancer or primary peritoneal cancer.

In one embodiment of the present invention, the all-trans retinoic acid inhibits the growth of cancer cell having stemness and tumourigenic characteristics.

In one embodiment of the present invention, the all-trans retinoic acid inhibits the growth of cancer cell by regulating ALDH1 expression.

According to the features above, the method of the present invention can regulate ALDH1 in an effective way. Despite the advent of surgical cytoreduction and combination chemotherapy, the majority of patients will ultimately recur and will succumb to disease. This emphasizes the need for novel therapies aimed at targeting cancer cells most resistant to initial therapy. The success of combination of anti-angiogenesis agent-Avastin with chemotherapy provides an excellent example for the successful combination of drugs with different modes of action. In this invention, the method for treating solid malignancy provides a new choice in cancer treatment.

### BRIEF DESCRIPTION OF THE DRAWINGS

The present invention will be apparent to those skilled in the art by reading the following detailed description of a preferred embodiment thereof, with reference to the attached drawings, in which:

FIGS. 1A-1F show ALDH1 regulates stemness and tumourigenic ability in ovarian cancer cells. FIG. 1A shows ALDH1-Low and ALDH1-High cells were stained with an Aldefluor® assay kit and separated with a FACSAria cell sorter. A total of 1×10³ A2780 (upper panel) and CP70 (lower panel) cells were seeded into a 96-well plate to evaluate the cells' sphere formation ability (scale bar=200 μm). DEAB was used to inhibit the reaction of ALDH1 and also served as a negative control. FIG. 1B shows western blotting was performed to evaluate ALDH1, FoxM1, Notch1, Oct-4 and Nanog expression. The β-actin was used as a protein loading control. The histogram represents the relative fold change of protein expression (right panel). FIG. 1C shows the tumourigenic ability of A2780-Low and A2780-High cells was assessed by inoculating 1×10³ cells into NOD/SCID mice. FIG. 1D shows A2780 cells were transfected with the ALDH1 overexpression plasmid (A2780-ALDH1), and CP70 cells were transfected with the shALDH1 plasmid (CP70-shALDH1). A2780-ALDH1 (upper panel) and CP70-shALDH1 (lower panel) cells were seeded into 96-well plates to evaluate their sphere formation ability (scale bar=200 μm). FIG. 1E shows the expression of CSC markers was analyzed by western blotting, and the quantitative analysis is shown (right panel). FIG. 1F shows the tumourigenic ability of A2780-ALDH1 and CP70-shALDH1 cells was assessed by inoculating 1×10³ cells into NOD/SCID mice. Student's t-test was used for statistical analyses (*P<0.05).

FIGS. 2A-2D show ALDH1 regulates stemness and tumourigenic ability through the FoxM1 and Notch1 signalling pathways. FIG. 2A shows A2780, CP70, CP70-vector and CP70-shALDH1 cells were treated with a FoxM1 inhibitor (1 μM Thiostrepton) or a Notch1 inhibitor (10 μM DAPT) and were then evaluated in sphere formation assays. Representative images show spheres generated from single-cell cultures after 10 days. The lower panel depicts the relative sphere formation ratio (scale bar=200 μm). FIG. 2B shows the ALDH1, FoxM1 and Notch-1 expression levels were evaluated by western blotting and the relative fold changes in expression are also shown. FIG. 2C shows the sphere formation ability of CP70-shFoxM1 (clones 1 and 2) and A2780-FoxM1 (clones 1 and 2) cells treated with or without DAPT (scale bar=200 μm). FIG. 2D shows protein expression analysed by western blotting. V: vector. Student's t-test was used for statistical analyses (*P<0.05).

FIGS. 3A-3I show ATRA treatment reduces ALDH1 expression, sphere formation ability, cell migration and invasion, and tumour growth in ovarian cancer cells. FIG. 3A shows cells were treated with ATRA (10 μM) for 28 days, and cell lysates were collected for western blotting. The quantitative analysis is shown in the right panel. FIG. 3B shows ALDH1 enzyme activity was detected using an Aldefluor® assay kit. FIG. 3C shows the changes in sphere formation ability in response to 10 μM ATRA treatment in A2780 (upper panel) and CP70 (lower panel) cells (scale bar=200 μm). FIG. 3D shows representative images of migrating cells treated with or without ATRA in a Transwell assay. The histogram shows the number of migrated cells (scale bar=100 _{I}lm). FIG. 3E shows representative images of invading cells treated with or without ATRA in a Transwell assay. The histogram shows the number of invading cells (scale bar=100 μm). FIG. 3F shows the cell doubling time was measured using an MTT cytotoxicity assay after treatment with or without ATRA. FIG. 3G shows ALDH1-High cells (1×10⁴) were pretreated with vehicle (DMSO) or ATRA (10 μM) for 28 days, and cells were collected for tumourigenesis analysis using a xenograft model. N.D.: not detected. FIG. 3H shows ALDH1 immunohistochemical analysis of tumour tissue sections shows the downregulation of ALDH1 expression after ATRA pretreatment. FIG. 3I shows A2780-Low and CP70-Low cells were treated with DMSO, ATRA (10 μM), or paclitaxel (60 ng/ml) for 4 days. The effect of ATRA and paclitaxel treatments on the percentage of ALDH1-High cells is shown. Student's t-test was used for statistical analyses (*P<0.05).

FIGS. 4A-4F show ATRA treatment reduces ALDH1 expression, sphere formation ability and cell migration in ovarian cancer cells. FIG. 4A shows ES2 cells were separated by Fluorescence-activated cell sorting (FACS) into stem-like ALDH1-High and non-stem-like ALDH1-Low cell populations. The efficiency of sphere formation in response to ATRA (10 μM) treatment is shown (scale bar=200 μm). FIG. 4B shows western blot analysis of ALDH1 and FoxM1. Right panel: relative fold changes of ALDH1 and FoxM1 expression compared with controls. FIG. 4C shows ES2-High cells were stained using an Aldefluor® assay kit and sorted with a FACSAria cell sorter. Sphere formation in ES2-High cells after treatment with different concentrations of Thiostrepton or DAPT, and the relative fold changes in expression are shown (scale bar=200 μm). FIG. 4D shows western blots of ALDH1, FoxM1 and Notch-1 in ES2-High cells. The right panel shows the fold changes in expression relative to controls. FIG. 4E shows representative images of migrating cells treated with or without ATRA by Transwell evaluation. The histogram shows the number of migrated cells. FIG. 4F shows the cell doubling time was measured using an MTT cytotoxicity assay after treatment with or without ATRA.

FIGS. 5A-5D show ATRA inhibits tumour growth in NOD/SCID mice. FIG. 5A shows flowchart of ATRA treatment in a mouse xenograft model. Cell suspensions of 1×10⁶ A2780-High or CP70-High cells were inoculated subcutaneously into mice. FIG. 5B shows representative tumours and tumour volumes in ALDH1-High A2780 or CP70 cells. Student's t-test was used for statistical analyses (*P<0.05). FIG. 5C shows immunohistochemical staining for ALDH1 in tumour tissue sections (scale bar=100 μm). FIG. 5D shows model depicting ALDH1 as a mediator of ATRA-induced tumour growth suppression in ovarian cancer cells.

### DETAILED DESCRIPTION OF THE PREFERRED EMBODIMENT

The accompanying drawings are included to provide a further understanding of the invention, and are incorporated in and constitute a part of this specification. The drawings illustrate embodiments of the invention and, together with the description, serve to explain the principles of the invention.

Unless otherwise defined, all technical and scientific terms used herein have the same meaning as commonly understood by one of ordinary skill in the art to which this invention pertains. In the case of conflict, the present document, including definitions will control.

### Definition

The term “ALDH1” in the specification is a short-term of “aldehyde dehydrogenase 1”.

The term “ATRA” in the specification is a short-term of “all-trans retinoic acid”.

The term “CSC” in the specification is a short-term of “cancer stem-like cell”.

The term “DEAB” in the specification is a short-term of “diethylaminobenzaldehyde”, which is an ALDH inhibitor.

The term “EOC” in the specification is a short-term of “epithelial ovarian cancer”.

As used herein, “approximately” shall generally mean within 20 percent, preferably within 10 percent, and more preferably within 5 percent of a given value or range. Numerical quantities given herein are approximate, meaning that the term “approximately” can be inferred if not expressly stated.

### Materials and Methods

### Cell Culture

The human ovarian cancer cell lines ES2, A2780 and its cisplatin-resistant derivative CP70 were obtained from the American Type Culture Collection (Manassas, Va.). These cells were grown in RPMI-1640 medium with 10% foetal bovine serum. Cells were cultured and stored according to the supplier's instructions and were used between passages 5 and 20. Once resuscitated, the cell lines were regularly authenticated through cell morphology monitoring, growth curve analysis, species verification and contamination checks.

### Inhibitors

The ALDH inhibitor diethylaminobenzaldehyde (DEAB) was purchased from StemCell Technologies (Vancouver, BC, Canada). DAPT (N-[2S-(3,5-difluorophenyl)acetyl]-L-alanyl-2-phenyl-1,1-dimethylethyl ester-glycine; Cayman Chemical, Ann Arbor, Mich.) dissolved in dimethyl sulphoxide, was used to test the effect of Notch signalling blockade. The FoxM1 inhibitor thiostrepton and ATRA were purchased from Sigma (Sigma, St Louis, Mo.).

### MTT Cytotoxicity Assay

The cell lines were cultured in a humidified incubator containing 95% air and 5% CO₂ at 37° C. in 96-well flat-bottomed microtiter plates. After 72 h of incubation, the in vitro cytotoxic effects of treatments were determined by MTT assay (at 570 nm).

### Sphere Formation Assay

Standard sphere formation assays were performed according to Zhang et al. (Zhang, S. et al. Cancer Res., 68, 4311-4320.) with minor modification. The cells (1×10³) were resuspended in serum-free DMEM/F12 medium supplemented with 5 μg/ml insulin (Sigma), 20 ng/ml human recombinant epidermal growth factor (EGF; Invitrogen, Life Technologies, Carlsbad, Calif.) and 10 ng/ml basic fibroblast growth factor (bFGF; Invitrogen) in ultra-low attachment plates (Corning Costar, Corning, N.Y.). Spheres that arose within 1-2 weeks were counted. Colony diameters >50 μm were counted as a single-positive colony. The middle field was chosen for counting of spheres, and two fields for each plate were counted under a dissecting microscope. For all sphere formation experiments, a minimum of eight wells was run for each condition. All data represent the mean ±SEM of three separate experiments and at least 24 different fields.

In vivo Mouse Xenografts

All animal studies adhered to protocols approved by the Institutional Animal Care and Use Committee of National Cheng Kung University Medical Centre. The mouse xenograft model was prepared as previously reported (Molthoff, C. F. et al. (1991) Int. J. Cancer, 47, 72-79). Briefly, cells were implanted in 50% Matrigel (BD Biosciences, San Jose, Calif.) and injected subcutaneously into the left flanks of 4- to 6-week-old female NOD/SCID (NOD.CB17-PRKDC (SCID)) mice. We observed mice for tumour formation every other day after cell inoculation and measured tumour size when it became measurable. Tumour size was measured using Vernier callipers by external measurement of the length and width, and the tumour volume was computed from the formula for an ellipsoid body [volume=(length/2)×(width)]. When the tumours reached ˜2000 mm³ at approximately 28 days, the mice were sacrificed. To assess the antitumour effect of ATRA, when tumours reached 100-200 mm³, mice received vehicle or ATRA (0.05 or 0.1 mg/kg) once every other day. All tumours were excised, fixed in 10% neutral buffered formalin, and embedded in paraffin for histological assessment or shock-frozen in liquid N₂ and stored at −80° C. for further analysis.

### Transwell Migration and Invasion Assays

Cells (1×10⁵) were seeded on Transwell filters with a pore size of 8 μm (Corning Costar) and were allowed to migrate toward medium containing 10% FBS. After 8 h, the cells on the upper surface of the Transwell membrane were removed with a cotton swab, and the migrated cells (on the underside of the Transwell) were fixed and stained with methanol and Giemsa staining dye (Merck, Darmstadt, Germany). The invasion assay was conducted in the same manner as the migration assay, except that Matrigel (BD Biosciences) was used, the incubation time differed (24 h) and the number of cells added to the upper chamber was 2×10⁵ cells. Cell migration and invasion were quantified by counting the migrated cells in six random fields under a light microscope.

### Separation of ALDH1-Low and -High Cells

An Aldefluor® kit (StemCell Technologies) was used to assess ALDH activity in the ovarian cancer cell lines, as previously described (Saw, Y. T. et al. (2012) BMC Cancer, 12, 329.). In brief, 1×10⁶ cells were incubated in Aldefluor® assay buffer containing a 1.5 μM ALDH substrate for 30 min at 37° C. Each sample was treated with 50 μM of DEAB, and used as a negative control. Prior to analysis, cells were stained with 1 mg/ml of propidium iodide to evaluate their viability. The fluorescence intensity of the stained cells was analysed using a FACSAria cell sorter Flow Cytometer (BD Biosciences). The reaction with DEAB was used to define the baseline for the assay. The ALDH activity of a sample was determined to be ‘high’ or ‘low’ based on the fluorescence intensity beyond or below the threshold defined by the reaction with DEAB. The cells having high or low ALDH activity are marked “-High” or “-Low” hereafter.

### ALDH1 and FoxM1 Overexpression/Knockdown and Transfection

We generated stable cell lines (A2780-ALDH1, A2780-FoxM1, CP70-shALDH1 and CP70-shFoxM1) from A2780 and CP70 cells with plasmid vectors encoding ALDH1 and shALDH1. ALDH1 short hairpin RNA was prepared and maintained according to the protocol provided by the National RNAi Core Facility, Academia Sinica, Taipei, Taiwan. To establish stable clones, the ALDH1 knockdown plasmids (NM-000689, National RNAi Core Facility) were transfected into CP70 cells, and the ALDH1 overexpression plasmid (ALDH1-pcDNA3.1, Addgene, Cambridge, Mass.) was transfected into A2780 cells using Lipofectamine (Invitrogen). Forty-eight hours after transfection, stable sh-ALDH1 and sh-FoxM1 transfectants were selected with puromycin (Sigma) at 0.3 μg/ml, and stable ALDH1 and FoxM1 transfectants were selected in G418 (Sigma) at 600 μg/ml. After 2 weeks of selection in puromycin or G418, clones of resistant cells were isolated and allowed to grow in medium containing puromycin at 0.3 μg/ml or G418 at 600 μg/ml. The integration of transfected plasmid DNA was confirmed by reverse transcription-PCR and western blot analyses.

### RNA Isolation and Quantitative Reverse Transcription-PCR

Total RNA was isolated using Trizol reagent (Invitrogen). Reverse transcription and real-time PCR experiments were performed using a High Capacity cDNA Reverse Transcription Kit (Promega, Madison, Wis.) and SYBR® Green PCR kit, respectively (Invitrogen). The following primers were used to amplify the various ALDH isozymes: ALDH1: 5′-TCCTGGTTATGGGCCTACAG-3′ (forward; SEQ ID No. 1), 5′-CTGGCCCTGGTGGTAGAATA-3′ (reverse; SEQ ID No. 2); GAPDH: 5′-GACAGTCAGCCGCATCTTCT-3′ (forward; SEQ ID No. 3), 5′-TTAAAAGCAGCCCTGGTGAC-3′ (reverse; SEQ ID No. 4).

### Western Blotting

The cells were lysed and then harvested using a cell lifter (Corning Costar). Protein concentrations were determined by Bio-Rad protein assay (Bio-Rad, Hercules, Calif.). Proteins were then separated by 10% sodium dodecyl sulphate-polyacrylamide gel electrophoresis and transferred to polyvinylidene difluoride membranes. Western blotting was performed using the following antibodies at the indicated dilutions: anti-ALDH1 (1:1000; BD Biosciences), anti-Oct4 (1:1000; BD Biosciences), anti-Nanog (1:1000; BD Biosciences), anti-Notch1 (1:1000; Abcam, Cambridge, UK), anti-FoxM1 (1:1000; Santa Cruz Biotechnology, Santa Cruz, Calif.) and anti-beta actin (1:5000; Sigma).

### Immunohistochemical Staining

The paraffin-embedded sections (5 μm thick) were placed on silane-coated slides and processed for immunohistochemistry. Immunohistochemical staining was performed on deparaffinized tissue sections of formalinfixed materials after microwave-enhanced epitope retrieval, based on the standard automated immunohistochemical procedure (Ventana XT autostainer; Ventana Medical Systems, Tucson, Ariz.). Endogenous peroxidase activity was blocked using 3% hydrogen peroxide. The slides were incubated with primary mouse monoclonal antibody against ALDH1 (clone 44/ALDH, 1:200 dilution; BD Biosciences, San Jose, Calif.) for 60 min at room temperature. The anti-ALDH1 antibody was located using a cocktail of horseradish peroxidase-labelled secondary antibodies (the ultraView Universal DAB Detection Kit), containing biotin-free reagents to eliminate biotin background staining and to optimize specificity. The complex was then visualized with hydrogen peroxide substrate, 3,3′-diaminobenzidine tetrahydrochloride chromogen and hematoxylin II (Ventana) counterstain. A negative control was established by replacing the primary antibody with phosphate-buffered saline. Normal hepatic cells were used as positive controls.

### Statistical Analysis

Data were analysed using the Statistical Package for the Social Sciences, Version 17.0 for Windows (SPSS). Values in this study are represented as the means±standard deviation. Student's t-test and analysis of variance with Tukey's post-hoc test were used to test for differences between two groups and multiple comparisons, respectively. P<0.05 (two-sided) was considered significant.

### EXAMPLE 1

### ALDH1 Regulates Sternness in Ovarian Cancer Cells

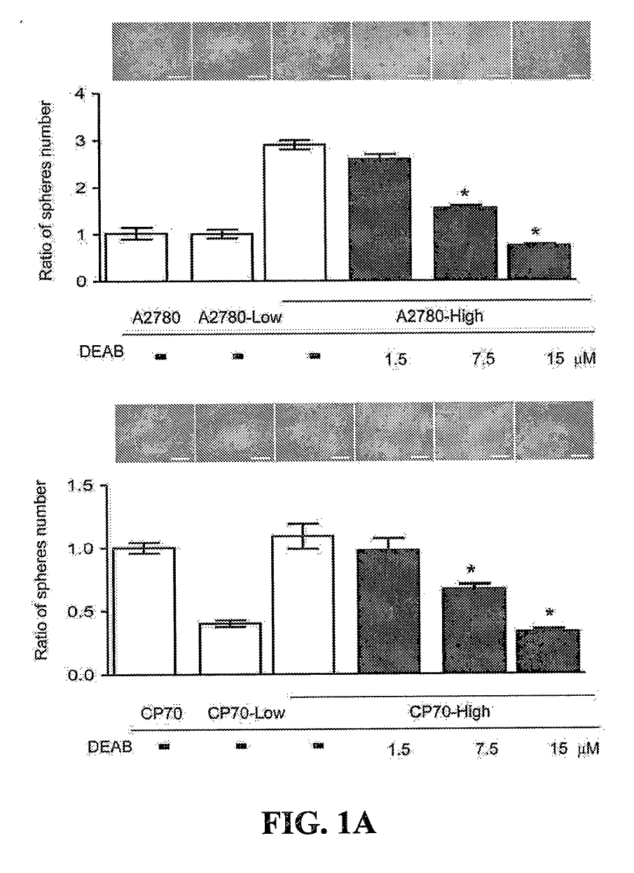

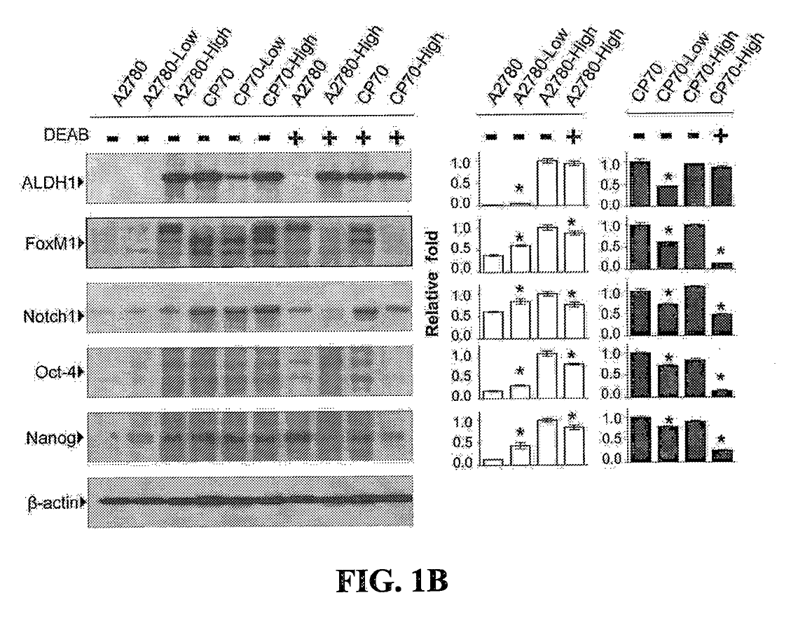

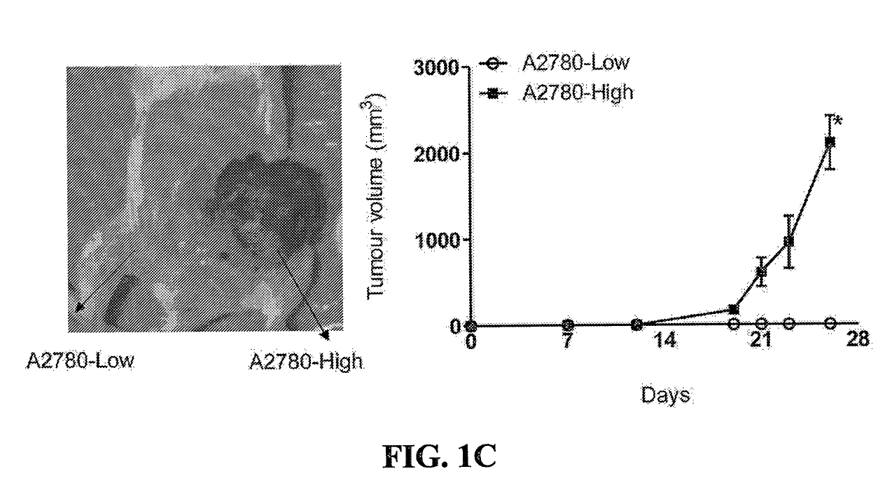

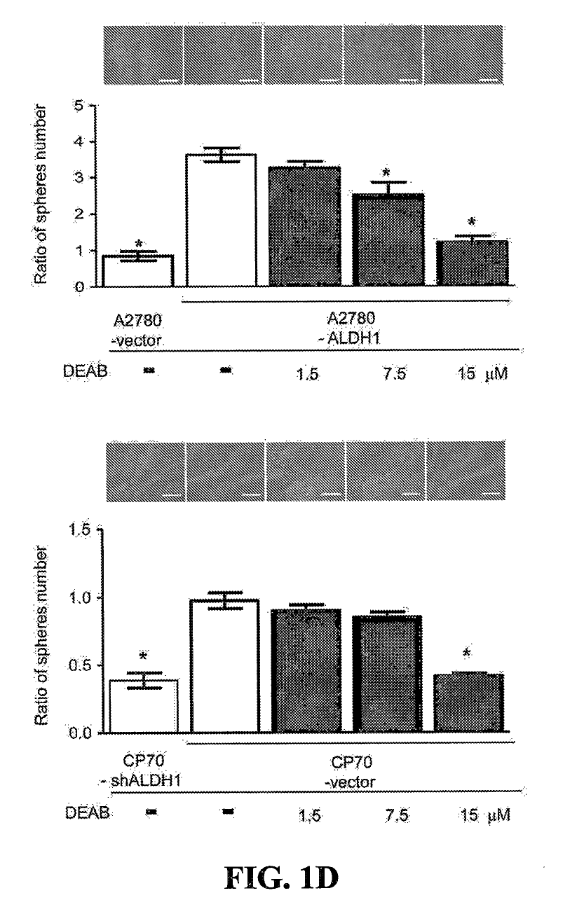

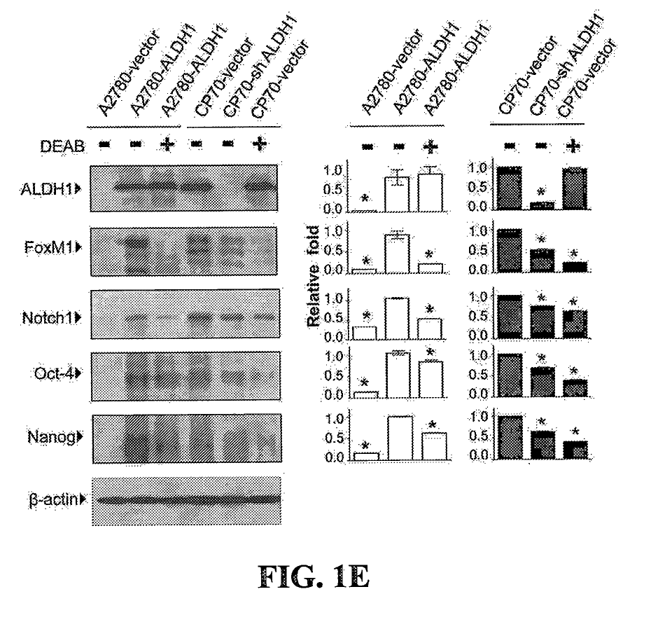

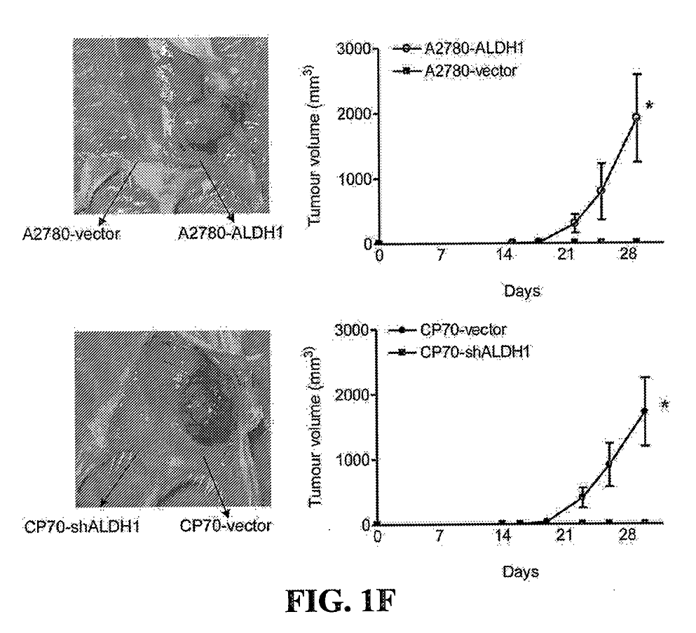

To evaluate whether the manipulation of ALDH1 activity can alter stemness and tumour formation in vivo, we enriched the endogenous population of cells expressing high ALDH1 activity by flow cytometry. Cells with high or low ALDH1 activity were termed ‘ALDH1-High’ or ‘ALDH1-Low’. The sphere formation efficiency of ALDH1-High A2780 or CP70 cells was significantly higher than that of their ALDH1-Low counterparts (FIG. 1A). DEAB, an ALDH1 activity inhibitor, suppressed sphere formation in ALDH1-High cells. The expression of CSC marker proteins such as FoxM1, Notch1, Oct 4 and Nanog was also inhibited by DEAB treatment (FIG. 1B). Moreover, DEAB treatment did not affect cell proliferation in these cells. We then tested in vivo tumourigenesis by inoculating 1×10³ cells subcutaneously into the mice, and we found that A2780-High cells had accelerated tumour formation ability compared with the A2780-Low cells (FIG. 1C). We then developed A2780-ALDH1 and CP70-shALDH1 cell lines that were stably transfected with ALDH1 and sh-ALDH1 complementary DNA. The ALDH1 RNA and protein levels in the aforementioned cells were highly correlated with the ALDH1 activity as measured by the Aldefluor® assay. The overexpression of ALDH1 resulting from transfection increased sphere formation efficiency (FIG. 1D, upper panel), induced upregulated expression of CSC markers (FIG. 1E) and enhanced tumour formation in a mouse xenograft model (FIG. 1F, upper panel). In contrast, ALDH1 silencing decreased sphere formation (FIG. 1D, lower panel), induced downregulation of CSC markers (FIG. 1E) and suppressed tumour formation (FIG. 1F). Collectively, our results indicate that ALDH1 contributes to stemness and tumourigenic ability in ovarian cancer cells.

### EXAMPLE 2

### FoxM1 and Notch1 are Involved in the Regulation of ALDH1-Mediated Sternness

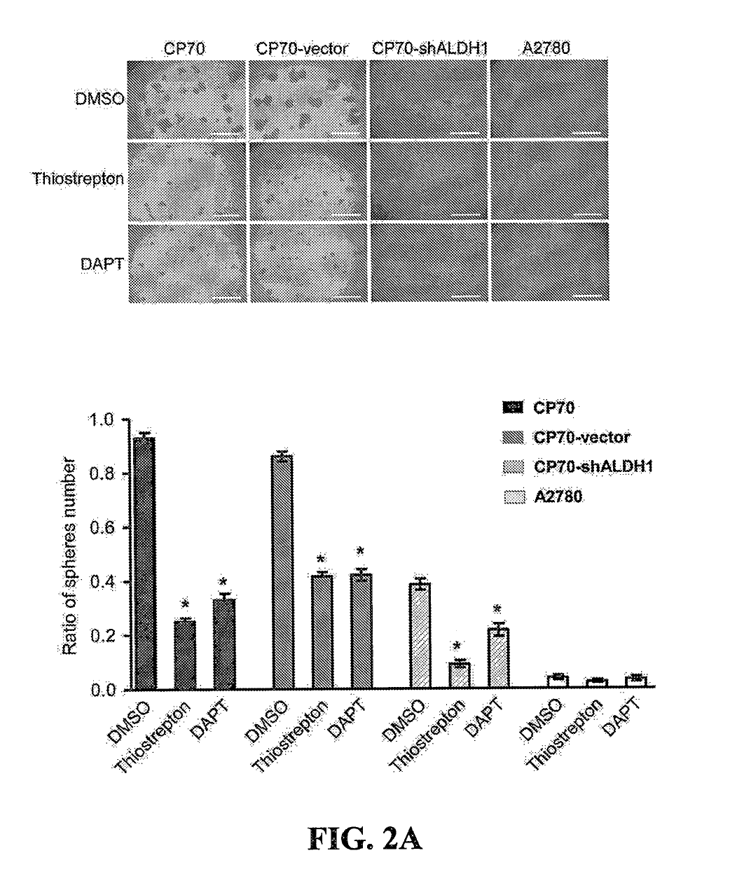

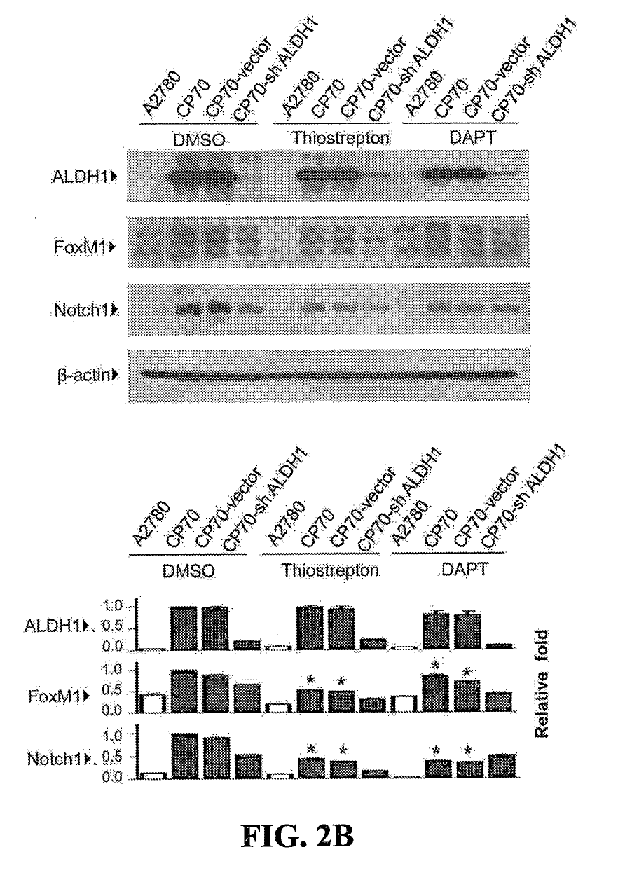

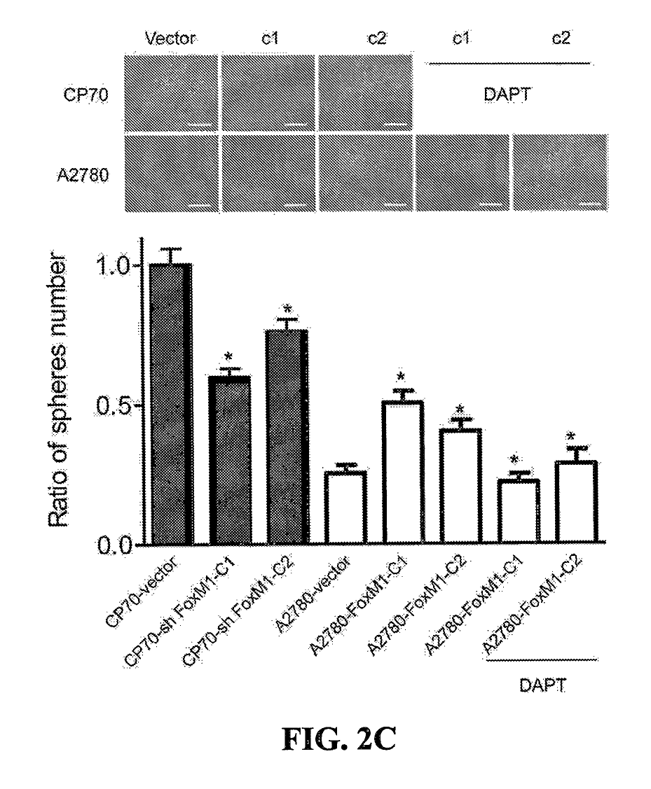

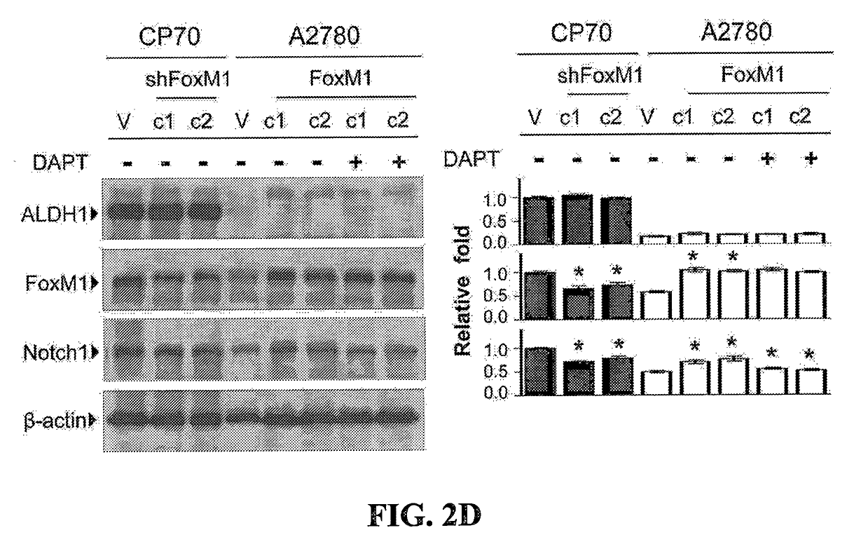

FoxM1 and Notch1 signalling have been reported to play a role in the biology of ovarian CSCs and to be involved in the pathophysiology of ovarian cancer. Because the expression levels of FoxM1 and Notch1 are concordant with that of ALDH1 (FIG. 1B and FIG. 1E), we tested whether FoxM1 and Notch1 signalling are involved in ALDH1-mediated stemness. As shown in FIG. 2A, sphere formation was decreased in shALDH1 compared with control cells. FoxM1 and Notch1 inhibition by Thiostrepton and DAPT decreased sphere formation efficiency in control and shALDH1 cells. DEAB downregulated the expression levels of both FoxM1 and Notch1 (FIG. 1B and FIG. 1E), whereas Thiostrepton and DAPT did not affect ALDH1 expression (FIG. 2B). DAPT treatment did not affect proliferation of these cells. Thiostrepton at 1 mM induced slight toxicity in A2780 and CP70-shALDH1 cells, but the effect was not significant. This finding indicated that ALDH1 regulates stemness through downstream FoxM1 and Notch1 signalling. Similar to the results observed in A2780 and CP70 cells, Thiostrepton reduced the expression not only of FoxM1 but also of Notch1, while DAPT decreased Notch1 expression as well as FoxM1 expression, but to a lesser extent (FIG. 2B). To further clarify the interaction between FoxM1 and Notch1 in ALDH1-regulated signalling, we generated CP70-shFoxM1 and A2780-FoxM1 cells for further analysis. FoxM1 silencing reduced sphere formation, whereas overexpression of FoxM1 increased sphere formation activity (FIG. 2C). In contrast to the results shown in FIG. 2B, immunoblot analysis showed that DAPT failed to attenuate FoxM1 expression in

FoxM1-overexpressing cells (FIG. 2D).

### EXAMPLE 3

### The Antitumour Effect of ATRA in Ovarian Cancer Cells

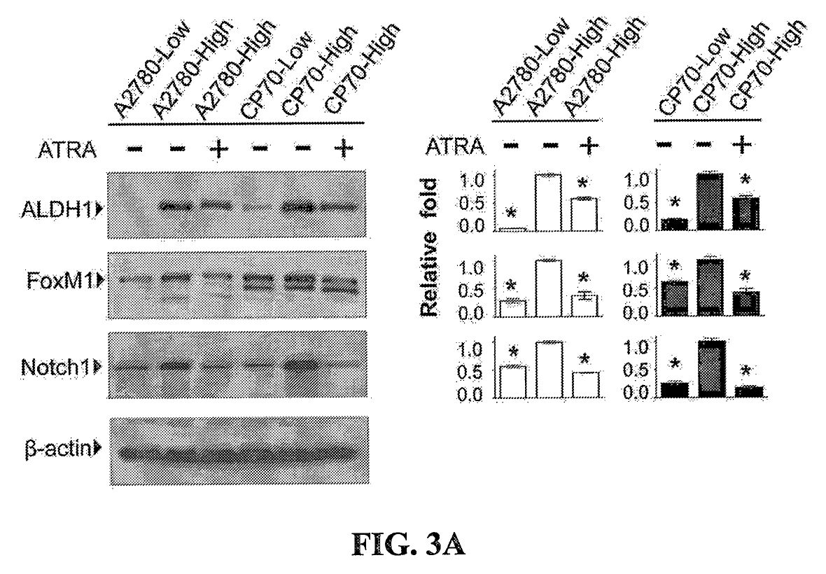

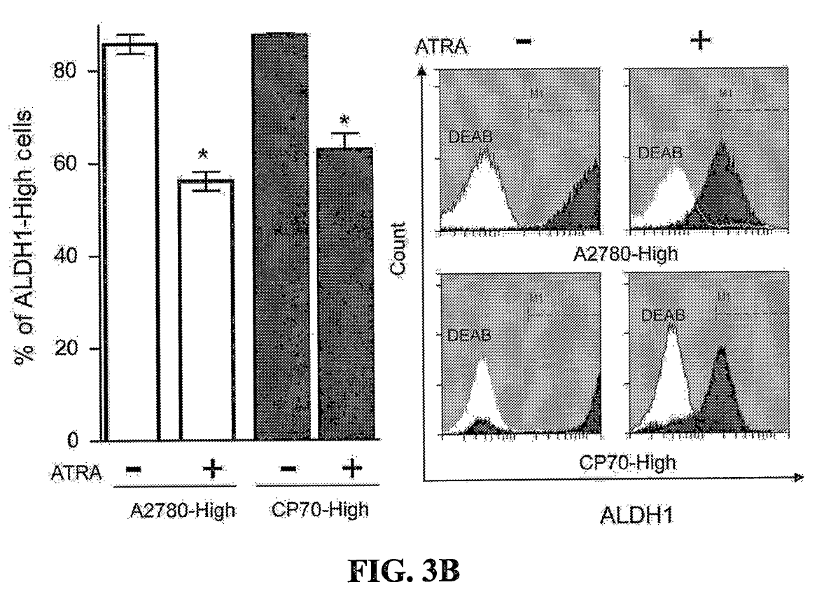

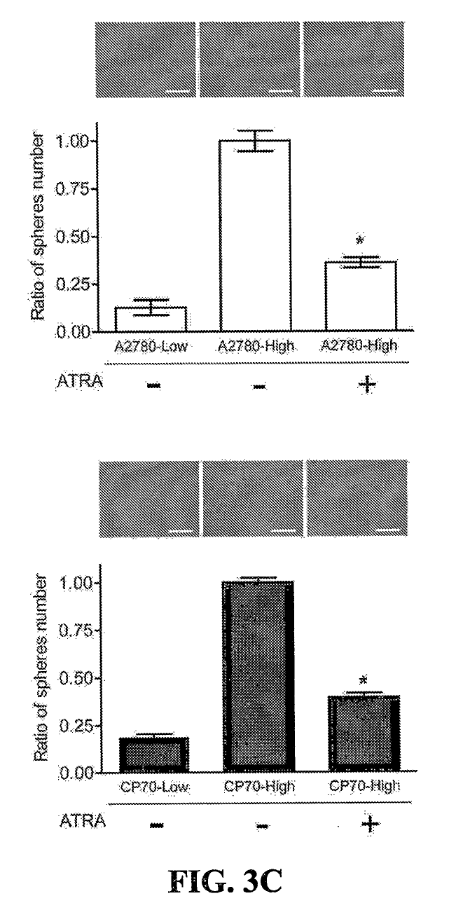

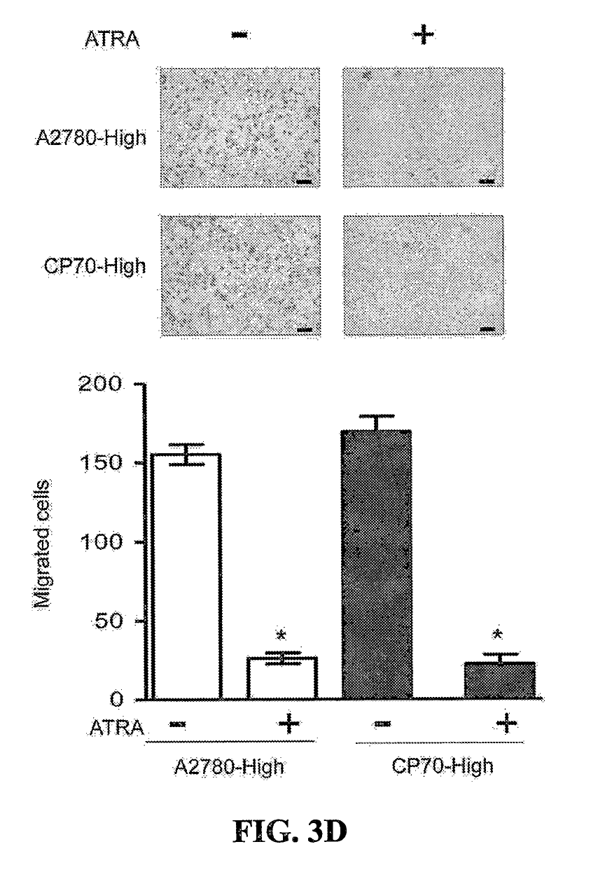

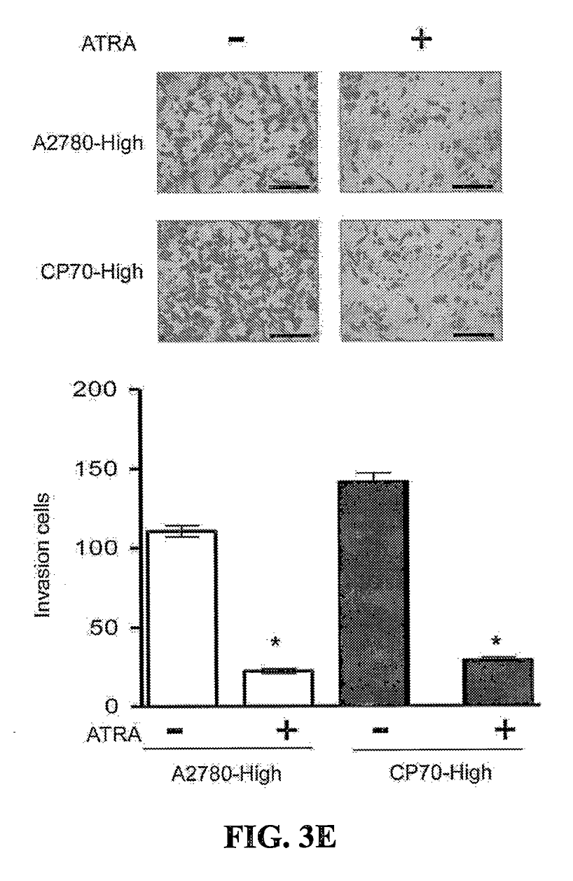

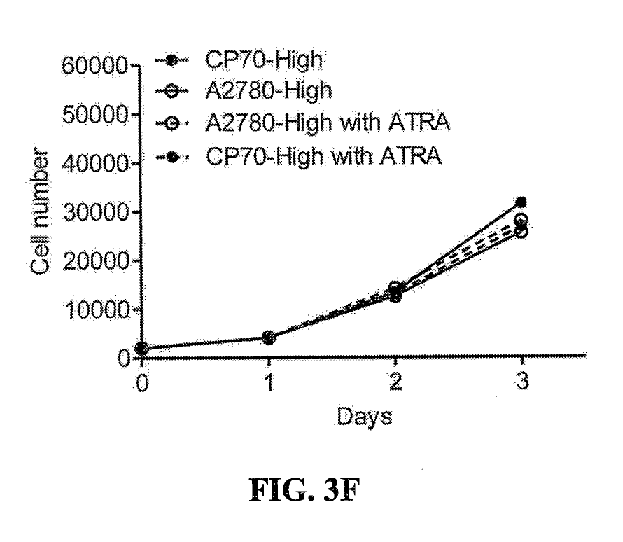

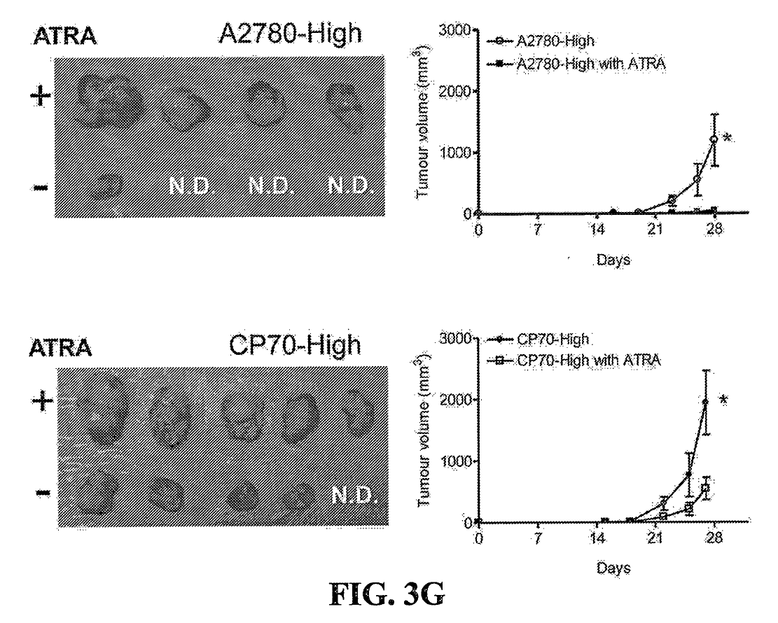

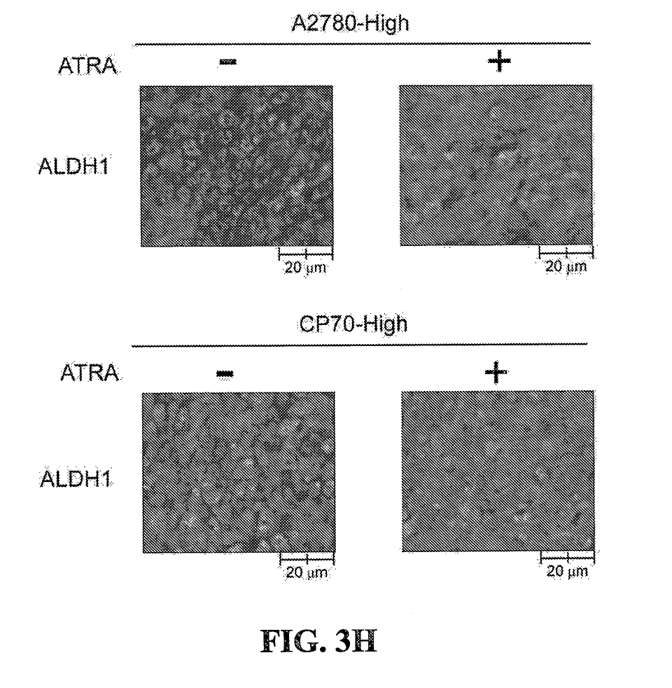

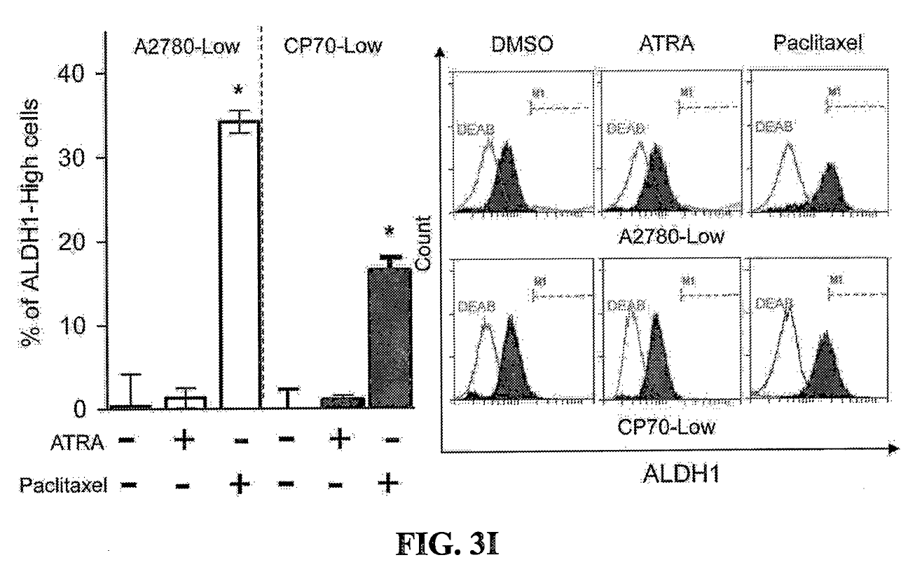

After pretreatment with 10 μM ATRA for 28 days, ALDH1, FoxM1 and Notch1 expression (FIG. 3A), ALDH1 activity (FIG. 3B), sphere formation ability (FIG. 3C) and cell migration and invasion abilities (FIG. 3D and FIG. 3E) were significantly reduced in A2780-High and CP70-High cells. ATRA treatment did not affect proliferation of these cells (FIG. 3F).

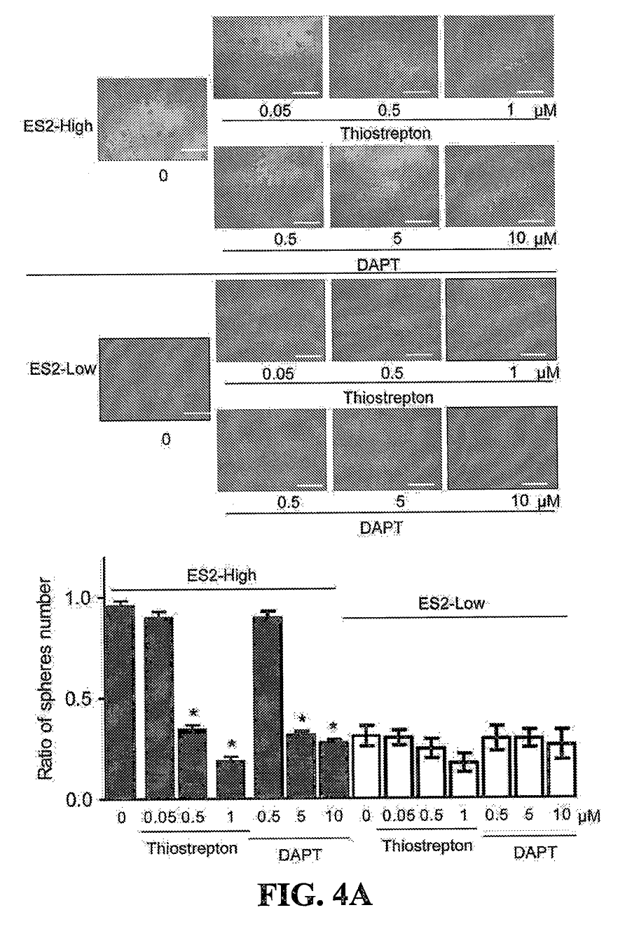

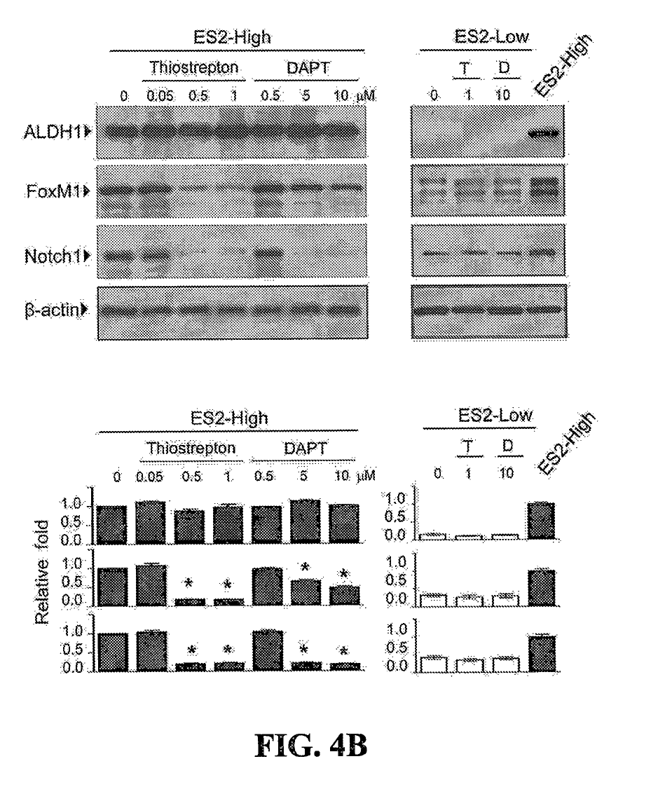

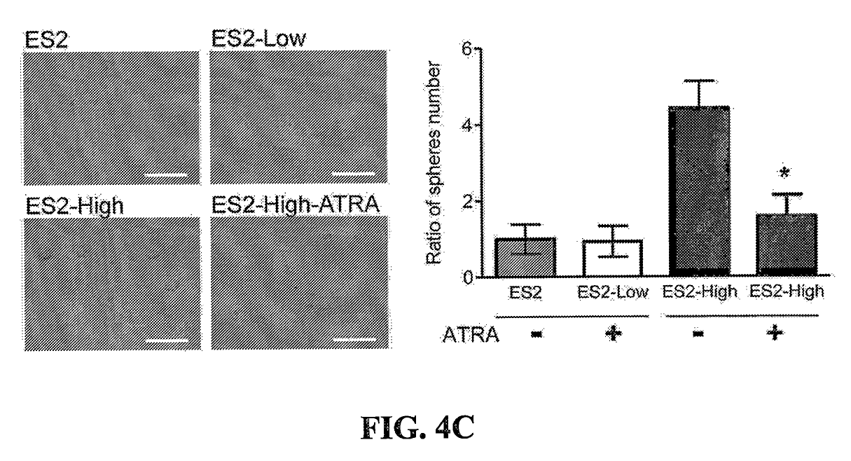

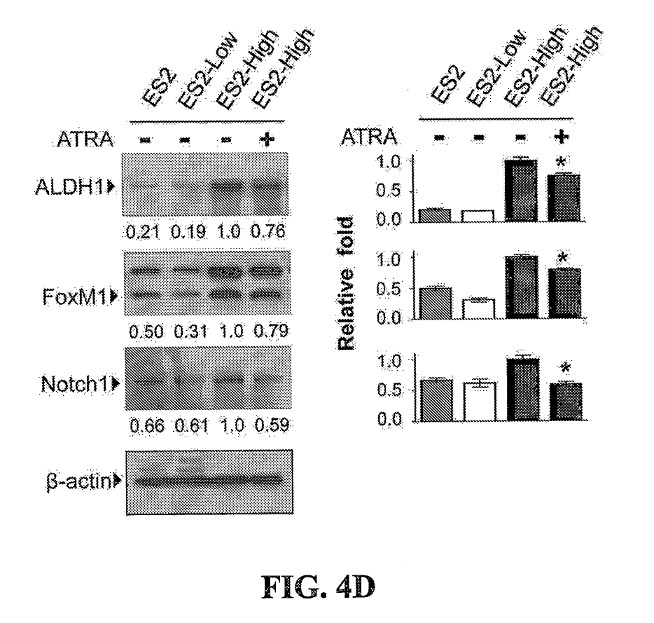

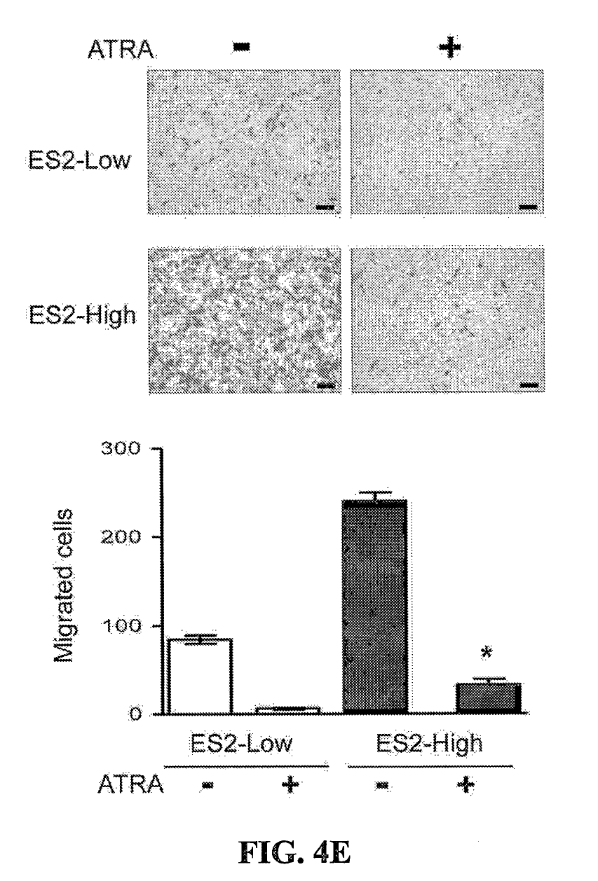

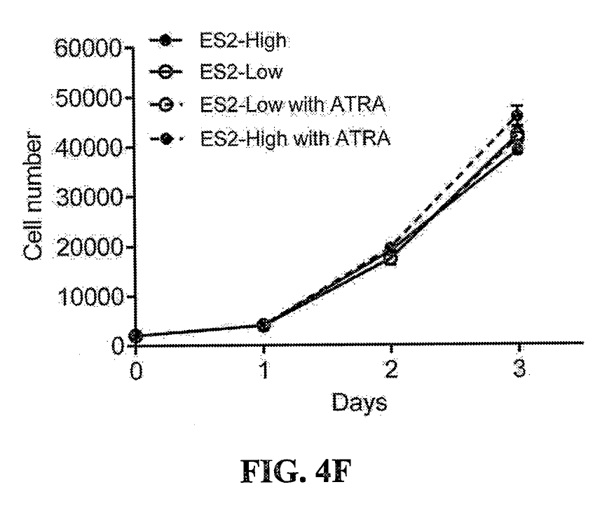

We then inoculated 1×10⁴ ATRA-pretreated A2780-High or CP70-High cells into mice. Tumour formation ability was almost completely abrogated in A2780-High cells, and a significant reduction of tumour size was also observed in CP70-High-innoculated mice (FIG. 3G). Immunostaining further confirmed that ATRA reduced ALDH1 expression in tumour cells (FIG. 3H). ATRA treatment decreased the proportion of ALDH1-High ovarian cancer cells (FIG. 3B and FIG. 3I); this finding is in contrast to the increased proportion of ALDH1-High cells resulting from treatment with chemotherapeutic agents such as paclitaxel (FIG. 3I). Similarly, Thiostrepton and DAPT decreased sphere formation in ES2-High cells in a dose-dependent manner (FIG. 4A). Thiostrepton and DAPT downregulated the expression levels of both FoxM1 and Notch1 in ES2-High cells in a dose-dependent manner, but did not affect ALDH1 expression (FIG. 4B). However, these findings were not observed in ES2-Low cells (FIG. 4A and FIG. 4B). ATRA inhibited sphere formation ability (FIG. 4C), ALDH1, FoxM1 and Notch1 expression (FIG. 4D) and cell migration ability (FIG. 4E) in ES2-High cells. ATRA treatment did not affect proliferation of these cells (FIG. 4F).

### EXAMPLE 4

### Antitumour Efficacy of ATRA in Mouse Xenografts

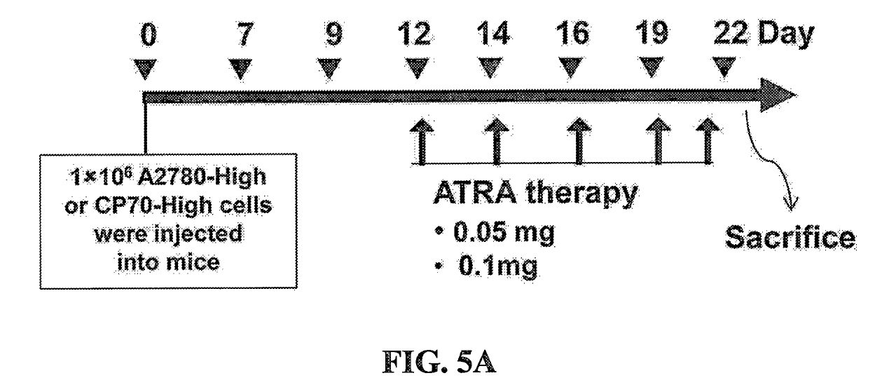

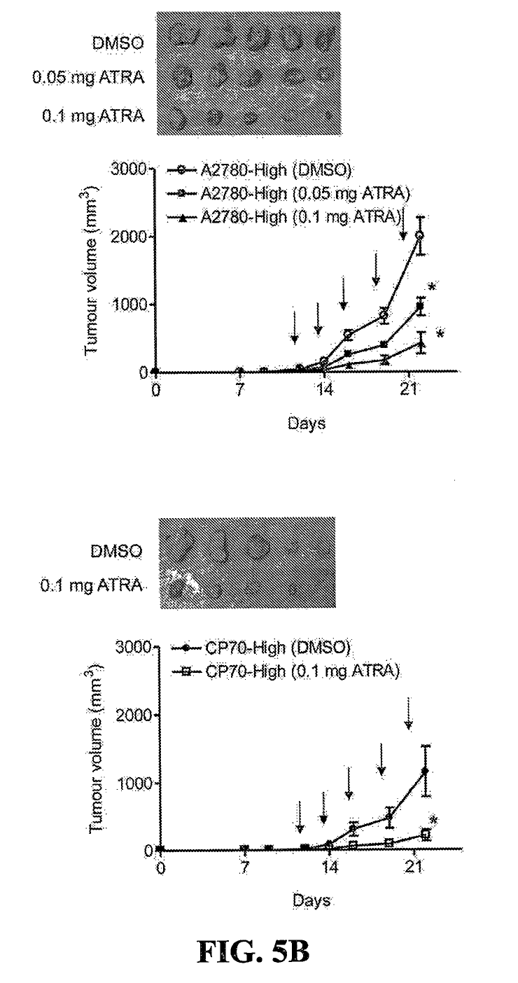

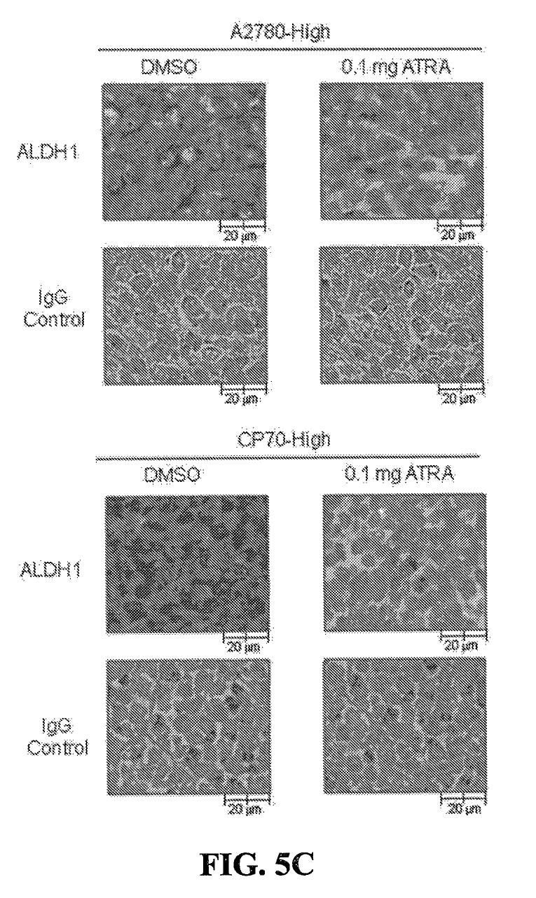

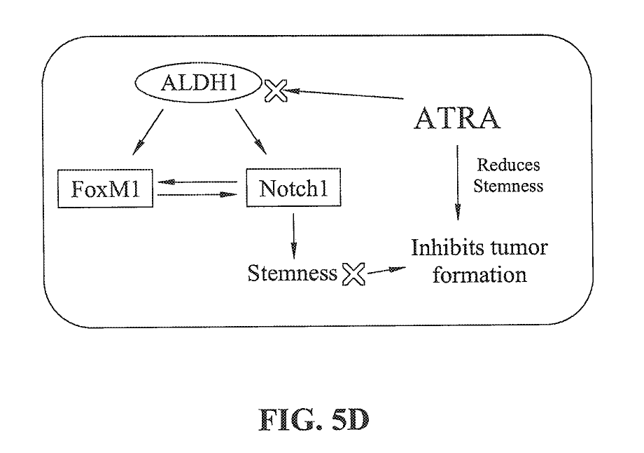

Cell suspensions of 1×10⁶ A2780-High or CP70-High cells were inoculated subcutaneously into mice. ATRA (0.05 or 0.1 mg) was injected into the peritoneal cavity three times per week, as illustrated in FIG. 5A. ATRA treatment inhibited A2780-High tumour growth in a dose-dependent manner and significantly suppressed CP70-High tumour growth at 22 days after tumour cell inoculation (FIG. 5B). ALDH1 expression in tumour cells from tissue sections was downregulated in ATRA-treated mice compared with control mice (FIG. 5C). These results further confirmed that ATRA can target ALDH1 and reduce the oncogenic potential of ALDH1-abundant cells.

In the above embodiments of the present invention, we investigated the importance of ALDH1 and the therapeutic role of ATRA in ovarian cancer cells. The principal finding of our study was that ALDH1 is a key player in regulating stemness and tumour formation in ovarian cancer cells; this regulation occurs through the downstream signalling of FoxM1/Notch1. In addition, ATRA downregulates ALDH1/FoxM1/Notch1 signalling and suppresses sphere formation ability, cell migration and invasion and tumourigenesis.

ALDH1 is not only a stem cell marker but also directly regulates the functions of ovarian cancer cells. ALDH1 expression was closely associated with tumourigenic potential in various ovarian cancer cell lines (Table 1), and FoxM1 and Notch1 were found to be important downstream effectors for ALDH1-regulated cancer stemness in ovarian cancer cells.

TABLE 1
Correlation between ALDH1 expression and tumourigenic potential in
ovarian cancer cells^{a}

| | Number of tumours/number of injections | | | | | | |
|---|---|---|---|---|---|---|---|
| | (average tumour volume, mm³) | | | | | Tumour-initiating cell frequency | |
| No. of cell injected | 1 × 10³ | | 1 × 10⁴ | | 1 × 10⁶ | (95% confidence interval) | |
| A2780 | 0/4 | (0) | 0/4 | (0) | 4/4 (1082) | 1/221 200 | (1/33 963-1/440 684) |
| A2780-vector | 0/5 | (0) | | | 1/1 (1344) | 1/188 562 | (1/1559-1/22 799 963) |
| A2780-ALDH1 | 4/5 | (1910) | | | 1/1 (1479) | 1/621 | (1/209-1/1847) |
| A2780-Low | 0/5 | (0) | | | 2/2 (1002) | 1/166 835 | (1/2305-1/12 075 235) |
| A2780-High | 5/5 | (1980) | 8/9 | (1115) | 2/2 (1400) | 1/2 267 | (1/841-1/6110) |
| A2780-High-ATRA | | | 4/9 | (109) | | 1/17 013 | (1/6295-1/45 976) |
| CP70 | 5/5 | (1014) | 4/4 | (1036) | 4/4 (1568) | 1/1 | (1/1-1/1254) |
| CP70-vector | 5/5 | (1716) | | | 2/2 (1906) | 1/1 | (1/1-1/1255) |
| CP70-shALDH1 | 0/5 | (0) | | | 2/2 (1419) | 1/166 835 | (1/2305-1/12 075 235) |
| CP70-High | | | 5/5 | (1937) | | 1/1 | (1/1-1/12 548) |
| CP70-High-ATRA | | | 4/5 | (545) | | 1/6213 | (1/2091-1/18 466) |

^{a}The frequency of ovarian-initiating cells was calculated by uploading the data into the web-based ELDA (Extreme Limiting Dilution Analysis) statistical software at http://bioinf.wehi.edu.au/software/elda/index.html.

FoxM1 affects the expression and function of a variety of genes that are critical to cell proliferation and survival, invasion, angiogenesis, and self-renewal of cancer stem cells. Genome-wide gene expression profiling of cancers has identified. Thiostrepton suppressed the expression of FoxM1 and Notch1, and the Notch1 inhibitor DAPT suppressed FoxM1 expression in addition to that of Notch1 (FIG. 2B and FIG. 4B). Thiostrepton has been shown to inhibit FoxM1 and Notch1 activity; combined with our results, this suggests that the downregulation of Notch by DAPT inhibited FoxM1 expression, which is in agreement with prior findings in prostatic cancer cells.

We identified a novel role of ATRA for inhibition of stemness via ALDH1-regulated signaling in ovarian cancer. ATRA treatment decreases the proportion of ALDH1-positive cancer cells; this result implies that ATRA can target the stem-like ALDH1-positive cell population. This finding is in contrast to the known tendency of chemotherapeutic agents, such as paclitaxel, to target the non-stem-like ALDH1-negative cell population; paclitaxel treatment thus increases the proportion of ALDH1-positive CSCs. The antitumour effect of ATRA, achieved by targeting the self-renewal pathways (ALDH1/FoxM1/Notch1) of ovarian cancer cells, indicates that ATRA might have therapeutic applications via inhibition of tumour behavior in ALDH1-expressing cancer cells or CSCs. Our findings also implicate the involvement of FoxM1/Notch1, further suggesting that the inhibition of the ALDH1/FoxM1/Notch1 signalling pathways by ATRA or other agents might provide new opportunities for therapeutic intervention.

In the above embodiments of the present invention, we prove that ATRA can regulate ALDH1/FoxM1/Notch1 signalling pathways. As describe set forth, FoxM1 as one of the most commonly overexpressed genes in solid tumours. ATRA therefore can treat the solid tumours through regulating FoxM1. Further, tubal, ovarian, and primary peritoneal carcinomas have a common origin, pathogenesis, behavior and clinical therapeutics. Thus, ATRA can inhibit these cancer cells through the ALDH1/FoxM1/Notch1 signalling pathways.

In the above embodiments, we also examined antitumour efficacy of ATRA in mouse xenografts. Among the administered dose 0.05 and 0.1 mg, we found ATRA inhibited tumour cell growth in a dose-dependent manner, that is, the effective dose for mice is at least 0.05 mg. The human equivalent dose for 0.05 mg in mice is 0.00405 mg/kg (conversion factor=0.081).

Although the present invention has been described with reference to the preferred embodiments thereof, it is apparent to those skilled in the art that a variety of modifications and changes may be made without departing from the scope of the present invention which is intended to be defined by the appended claims.

1. A method for regulating aldehyde dehydrogenase 1 (ALDH1) comprises administering an effective dose of all-trans retinoic acid to a subject.

2. The method as claimed in claim 1, wherein the all-trans retinoic acid further regulates FoxM1 expression through regulating ALDH1.

3. The method as claimed in claim 1, wherein the all-trans retinoic acid further regulates Notch1 expression through regulating ALDH1.

4. The method as claimed in claim 1, wherein the subject is a mammal.

5. The method as claimed in claim 4, wherein the mammal is a human body.

6. The method as claimed in claim 5, wherein the effective dose is at least 0.00405 mg/kg.

7. A method for treating solid malignancy comprises administering an effective dose of all-trans retinoic acid to a subject; wherein the solid malignancy is ovarian cancer, fallopian tube cancer or primary peritoneal cancer.

8. The method as claimed in claim 7, wherein the subject is a mammal.

9. The method as claimed in claim 8, wherein the mammal is a human body.

10. The method as claimed in claim 9, wherein the effective dose is at least 0.00405 mg/kg.

11. (canceled)

12. The method as claimed in claims 7, wherein the all-trans retinoic acid inhibits the growth of cancer cell having sternness and tumourigenic ability.

13. The method as claimed in claim 7, wherein the all-trans retinoic acid inhibits the growth of cancer cell by regulating ALDH1 expression.

14. The method as claimed in claim 13, wherein the all-trans retinoic acid inhibits the growth of cancer cell by downregulating ALDH1 expression.


SEQUENCE LISTING (4 sequences)

SEQ ID NO: 1 - DNA, 20 bp, Homo sapiens
tcctggttatgggcctacag

SEQ ID NO: 2 - DNA, 20 bp, Homo sapiens
ctggccctggtggtagaata

SEQ ID NO: 3 - DNA, 20 bp, Homo sapiens
gacagtcagccgcatcttct

SEQ ID NO: 4 - DNA, 20 bp, Homo sapiens
ttaaaagcagccctggtgac